(19) United States
(12) Patent Application Publication
(10) Pub. No.: US 2026/0264147 A1
(43) Pub. Date: Sep. 10, 2026

(54) ADDITIVE MANUFACTURING OF A MICROSTRUCTURE

(71) Applicant: KONINKLIJKE PHILIPS N.V., EINDHOVEN (NL)

(72) Inventors: NAVEED UR RAHMAN, BEST (NL); JORRIT JORRITSMA, VELDHOVEN (NL); THOMAS HAFKAMP, BEST (NL); JAN GRAGE, HAMBURG (DE); RON DE KLOET, BEST (NL)

(21) Appl. No.: 19/153,717

(22) PCT Filed: Jan. 31, 2024
(86) PCT No.: PCT/EP2024/052310

§ 371 Date: Aug. 5, 2025

(30) Foreign Application Priority Data
Feb. 7, 2023 (EP) 23155414.8

(51) Int. Cl. B22F 10/28; B22F 10/50; B33Y 10/00; B33Y 40/00; B33Y 80/00; G21K 1/00

(52) U.S. Cl. CPC B22F 10/28; B22F 10/50; B33Y 10/00; B33Y 40/00; B33Y 80/00; G21K 1/00; B22F 2304/10; B22F 2998/10; B22F 2999/00

(57) ABSTRACT

The invention relates to a method for additive manufacturing of a microstructure (10) for selective transmission of X-ray or gamma-ray radiation. The method includes depositing (110) a layer of powder on a build plate or on a layer from at least one previous manufacturing step on the build plate, wherein the layer of powder comprises a high-Z material powder. Furthermore, the method includes selectively melting (120) and solidifying the powder to form septa wall structures (20) by scanning an electron beam in a spot sequence on the layer of powder to sequentially expose a plurality of spots (40) on the layer of powder to an electron beam spot. A size of the electron beam spot is at most 150 μm in diameter, preferably at most 75 μm in diameter, and more preferably at most 50 pm in diameter. The high-Z material powder comprises a high-Z material having an atomic number Z of at least 40, and preferably at least 70. The invention also relates to a microstructure for selective transmission of X-ray or gamma-ray radiation, which microstructure has been obtained by the method, and to an imaging component comprising such microstructures. The invention also relates to a method for manufacturing an imaging component.

### FIELD OF THE INVENTION

The invention relates to a method for additive manufacturing, more specifically to a method for additive manufacturing a microstructure for selective transmission of X-ray or gamma-ray radiation. The invention also relates to a microstructure for selective transmission of X-ray or gamma-ray radiation, which microstructure has been obtained by the method, and to an imaging component comprising such microstructures. The invention also relates to a method for manufacturing an imaging component.

### BACKGROUND OF THE INVENTION

Additive manufacturing technologies such as direct metal laser sintering, or laser powder bed fusion, show great promise for manufacturing of X-ray and gamma ray transmission selective microstructures like anti-scatter devices, 2D anti-scatter grids etc. In some cases, such as for microstructures with high density, high purity and/or low micro-cracking, other manufacturing technologies may be needed.

US2021/0039322A1 describes a method for providing a control command set for an additive manufacturing device. The method includes providing a parameter set consisting of a number of parameters, and a construction rule, which is suitable for describing at least one section of the object by the parameter set geometrically as a number of linear or flat elements in space; generating a computer-based layer model of the section of the object by determining, for each layer, the position and shape of a cross-section of the section of the object within the layer, generating a control command set for an additive manufacturing device by which the production of the section of the object is implemented on the basis of the layer model.

WO2022/248519A1 provides methods and devices for making an anti-scatter grid for a radiographic imaging device identifiable. A method for providing an anti-scatter grid for a radiographic imaging device comprises forming, by an additive manufacturing process, a grid pattern in accordance with a product specification of the anti-scatter grid to be provided; and forming, by an additive manufacturing process, a number of structural modifications in or at the grid pattern in a manner making the number of structural modifications image-based recognizable when the anti-scatter grid is viewed according to its intended use in a viewing direction from a radiation source of the radiographic imaging device.

Electron beam powder bed fusion (E-PBF) uses an electron beam to selectively melt a metal powder into solid material in a vacuum chamber. E-PBF can result in lower residual material stresses and reduced cracks thanks to higher build temperatures. E-PBF is commonly used to build microstructures with materials such as titanium-aluminum, cobalt-chrome, or nickel-based alloys.

Research Disclosure #697065 suggests feasibility to additively manufacture metal parts in refractory metals such as tungsten and alloys thereof, using E-PBF.

US2015/0017013A1 describes a method of manufacturing a turbo-machine impeller, which includes a hub and a plurality of blades, using powder material in an additive-manufacturing process. The method includes applying energy to the powder material by way of a high energy source, and solidifying the powder material. At least one bulky portion of the hub is irradiated such that the powder material solidifies in a lattice structure surrounded by an outer solid skin structure enclosing the lattice structure.

The publication “State of the arts of additive manufacturing by selective electron beam melting”, Mladenov Georgi et al, 5 Jun. 2016, reviews applications of electron beam additive manufacturing systems.

Known E-PBF approaches in the literature are not suitable for manufacturing of X-ray and gamma ray transmission selective microstructures, such as anti-scatter grids, since this kind of transmission selective microstructures may advantageously have high surface quality, high resolution, high septa wall density etc. Hence, there is a need to improve the manufacturing of these microstructures.

### SUMMARY OF THE INVENTION

It is an object of the invention to provide improved manufacturing of microstructures for selective transmission of X-ray or gamma-ray radiation.

The invention is defined by the independent claims. Advantageous embodiments are defined in the dependent claims.

According to a first aspect of the invention, there is provided a method for additive manufacturing of a microstructure for selective transmission of X-ray or gamma-ray radiation. The method comprises:
depositing a layer of powder on a build plate or on a layer from at least one previous manufacturing step on the build plate, wherein the layer of powder comprises a high-Z material powder; and
selectively melting and solidifying the powder to form septa wall structures by scanning an electron beam in a spot sequence on the layer of powder to sequentially expose a plurality of spots on the layer of powder to an electron beam spot, wherein a size of the electron beam spot is at most 150 μm in diameter, preferably at most 75 μm in diameter, and more preferably at most 50 μm in diameter.

The proposed manufacturing method enables high productivity of microstructures with high density septa walls for selective transmission of X-ray or gamma-ray radiation. The structures may be built without support structures and have little or no micro-cracking and excellent surface properties compared to previously known manufacturing technologies. Generally, it is known that a high electron beam density for powder bed spot melting may cause melting issues such as charging and smoke events. However, the inventors surprisingly found that in combination with a high-Z material powder it is advantageous to use a very small electron beam spot size, and thus, for a given electron beam power, a very high beam energy density for spot melting of the septa wall structures. Thanks to the very high material density of a high-Z material, in combination with the small electron beam spot size, the method enables high resolution manufacturing of the microstructures. Thereby the method enables desired local to global functional characteristics of microstructures for selective transmission of X-rays or gamma rays, such as anti-scatter grids. The current of the electron beam may be e.g. but not limited to 10-100 mA and the scanning speed e.g. up to or even larger than 2 m/s.

A high-Z material is a material (element) with a high atomic number Z. The high-Z material is preferably a metal. The high-Z material may preferably be Tungsten (Wolfram, W, with atomic number 74). Other high-Z materials, such as but not limited to, Molybdenum (Mo, atomic number 42), Tantalum (Ta, atomic number 73), Niobium (Nb, atomic number 41), Lead (Pb, atomic number 82), Bismuth (Bi, atomic number 83), Rhenium (Re, atomic number 75), Silver (Ag, atomic number 47) or Gold (Au, atomic number 79) may alternatively or additionally or in combination be used. So, in the context of this disclosure, a high-Z material has an atomic number Z of at least 40, and preferably at least 70. The high-Z material powder may alternatively or additionally or in combination comprise an alloy including at least one high-Z material.

According to an embodiment of the invention, the high-Z material powder has a particle size distribution with a median diameter of at most 50 μm, preferably with a median diameter of at most 25 μm, most preferably at most 20 μm. Despite the very high energy density of the beam, it was found that optimal results may be achieved with a powder having a small particle size distribution.

According to an embodiment of the invention, the deposited layer of powder has a layer thickness between 10 μm and 50 μm; the boundaries 10 μm and 50 μm are included within the scope of the claim. Thin layers of the powders, e.g. in combination with small particle size and the narrow electron beam, enable high resolution manufacturing of high density septa walls.

According to an embodiment of the invention, the method further comprises selectively pre-heating spots of the powder by scanning the electron beam in a spot pre-sintering sequence on the layer of powder, and wherein the spot pre-sintering sequence is carried out before the spot sequence. By pre-heating or pre-sintering spots of powder with the electron beam, subsequent melting of the powder with the spot-sequence can be improved.

According to an embodiment of the invention, a scanning direction and sequence of spots of the pre-sintering sequence is the same as a scanning direction and sequence of spots of the spot sequence for all spots, or, a scanning direction and sequence of spots of the pre-sintering sequence is the same as a scanning direction and sequence of spots of the spot sequence for a plurality of spots. Pre-heating and melting the powder according to the same or partly the same pattern, may be advantageous for the formation of septa walls with high density.

According to an embodiment of the invention, the septa wall structures of the microstructure comprise first wall structures formed with each a respective length axis parallel to a first direction and second wall structures formed with each a respective length axis parallel to a second direction, and wherein the second direction is at an angle to the first direction. With septa walls in two different directions at an angle, such as but not limited to e.g. 90 degrees or 60 degrees or 45 degrees, it is possible to advantageously manufacture grid microstructures, such as radiation anti scatter grids.

According to an embodiment of the invention, the spot sequence to sequentially expose a plurality of spots on the layer of powder to the electron beam spot comprises a first sequence followed by a second sequence, wherein the first sequence comprises scanning the electron beam spot to sequentially expose a plurality of spots on the length axes parallel to the first direction, and wherein the second sequence comprises scanning the electron beam spot to sequentially expose a plurality of spots on the length axes parallel to the second direction. This dedicated spot melting strategy with respect to the length axes of the formed septa walls may reduce the amount of sintered powder within a pixel of a grid to enable high resolution. Furthermore, the strategy may be used to achieve a desirable grain structure in each of the septa walls.

According to an embodiment of the invention, the first sequence comprises scanning the electron beam spot along a plurality of first septa wall paths on the respective length axes parallel to the first direction, and wherein the second sequence comprises scanning the electron beam spot along a plurality of second septa wall paths on the respective length axes parallel to the second direction. By scanning the electron beam spot first along septa wall paths corresponding to the first direction, and then along septa wall paths corresponding to the second direction it is possible to achieve advantageous manufacturing of septa walls in the respective directions. Scanning along a septa wall path in this context means that adjacent spots on the path are sequentially exposed to the electron beam spot, which thus moves along the path. It should be noted that the movement of the spot along a first septa wall path may be either parallel or anti-parallel to the first direction. Similarly, movement of the spot along a second septa wall path may be either parallel or anti-parallel to the second direction.

According to an embodiment of the invention, the septa wall structures have a wall thickness of at most 200 μm, preferably at most 100 μm, more preferably at most 70 μm.

According to an embodiment of the invention, the septa wall structures have a height to thickness aspect ratio of at least 100, preferably at least 200 and even more preferably at least 400.

Manufacturing of microstructures with thin septa walls, such as but not limited to septa walls with a thickness of at most 100 μm, and particularly septa walls with a very large height to thickness ratio, such as but not limited to thin septa walls with a height of at least 40 mm, may enable microstructures with precise control of transmission of X-ray or gamma-ray radiation. Such as, but not limited to, microstructures that efficiently reduce scattering of radiation, while having low losses in radiation intensity due to thickness of the walls.

According to a second aspect of the invention, there is provided a microstructure for selective transmission of X-ray or gamma-ray radiation, obtained by the method according to the first aspect or any embodiment thereof.

According to a third aspect of the invention, there is provided an imaging component comprising the microstructure.

According to an embodiment of the invention, the imaging component comprises a plurality of stacked microstructures. Stacking of multiple microstructures to form the imaging component may be advantageous for improved simplicity of manufacturing.

According to an embodiment of the invention, the imaging component comprises one or more of:
an X-ray or gamma-ray filter;
an X-ray or gamma-ray collimator;
an X-ray or gamma-ray anti-scatter device; and
an X-ray or gamma-ray grating.

According to a fourth aspect of the invention, there is provided a method of manufacturing an imaging component, wherein the method comprises manufacturing at least one microstructure according to the first aspect or any embodiment thereof, and forming the imaging component from the at least one microstructure. Forming the imaging component from at least one microstructure may include steps such as adjusting form factors, adding connections, adding layers or other components, modifying surfaces, integrating the microstructure in a holder or frame or similar structure, etc. The manufactured imaging component is suitable to be used in an imaging system.

According to an embodiment of the invention, manufacturing the imaging component comprises stacking a plurality of the microstructures. Stacking of multiple microstructures to form the imaging component may be advantageous for improved simplicity of manufacturing.

According to an embodiment of the invention, the imaging component comprises one or more of:
an X-ray or gamma-ray filter;
an X-ray or gamma-ray collimator;
an X-ray or gamma-ray anti-scatter device; and
an X-ray or gamma-ray grating.

These and other aspects of the invention will be apparent from and elucidated with reference to the embodiments described hereinafter.

### BRIEF DESCRIPTION OF THE DRAWINGS

FIG. 1 schematically illustrates a method for additive manufacturing of a microstructure for selective transmission of X-ray or gamma-ray radiation, according to an embodiment of the invention.

FIG. 2 schematically illustrates a method for additive manufacturing of a microstructure for selective transmission of X-ray or gamma-ray radiation, where the method includes a selective pre-heating step, according to an embodiment of the invention.

FIG. 3a and FIG. 3b schematically illustrate a microstructure for selective transmission of X-ray or gamma-ray radiation, according to an embodiment of the invention.

FIG. 4a and FIG. 4b schematically illustrate spot sequences for additive manufacturing of a microstructure for selective transmission of X-ray or gamma-ray radiation, according to an embodiment of the invention.

FIG. 5 shows optical surface images of a septa wall produced by a method of additive manufacturing according to an embodiment of the invention.

FIG. 6 shows an image of individual septa walls produced by a method of additive manufacturing according to an embodiment of the invention.

### DETAILD DESCRIPTION OF EMBODIMENTS

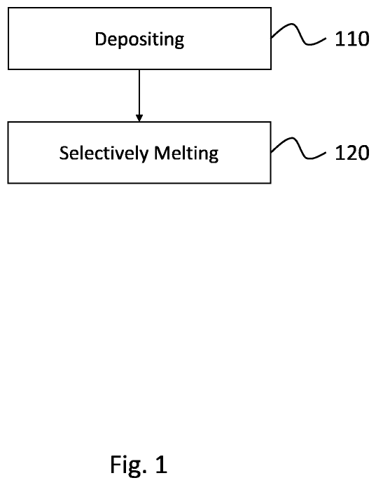

A method for additive manufacturing a microstructure 10 for selective transmission of X-ray or gamma-ray radiation, according to an embodiment of the invention, is illustrated with a flowchart in FIG. 1. The method in FIG. 1 includes a step of depositing 110 a layer of powder on a build plate or on a layer from at least one previous manufacturing step on the build plate, wherein the layer of powder comprises a high-Z material powder. The high-Z material may include e.g. Tungsten, Molybdenum, Tantalum, Niobium, Lead, Bismuth, Rhenium, Silver or Gold. Combinations of high-Z materials and/or alloys comprising at least one high-Z material may be used. As a non-limiting example, a high purity high-Z material powder may be used in combination with the method. In this way, it may be possible to achieve very high purity septa walls. It may be particularly advantageous to use a high-Z material powder with a purity (in weight % of high-Z material) above 99%, preferably above 99.9%. Such as e.g. a high purity Tungsten powder with a purity above 99%, preferably above 99.9%. The high-Z material powder may have a particle size distribution with a median diameter of at most 50 μm, preferably with a median diameter of at most 25 μm, most preferably at most 20 μm. Despite a very high energy density of the beam, such as but not limited to an electron beam with a beam current up to or even larger than 100 mA, it was found that optimal results may be achieved with a powder having a small particle size distribution. The deposited layer of powder may advantageously have a layer thickness between 10 μm and 50 μm.

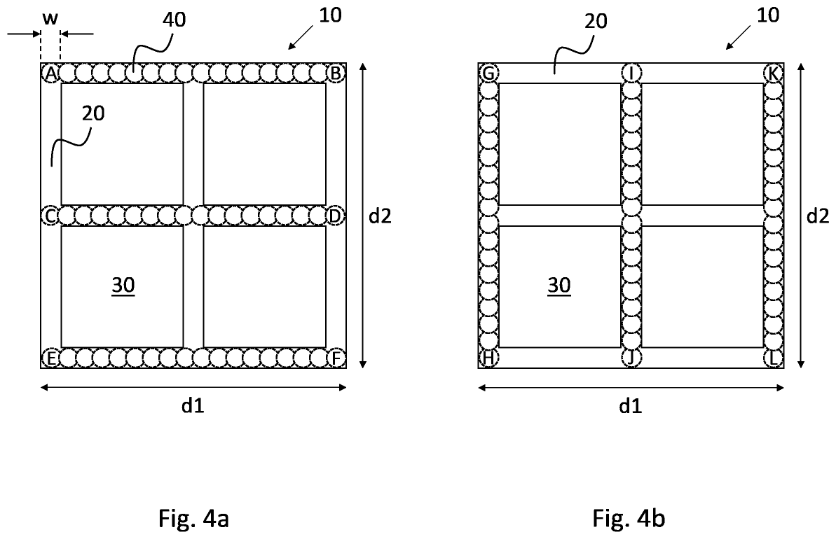

In a next step 120 of the method shown in FIG. 1, the layer of powder is selectively melted and solidified to form septa wall structures 20. This is achieved by scanning an electron beam in a spot sequence on the layer of powder to sequentially expose a plurality of spots 40, such as e.g. shown in FIG. 4a, on the layer of powder to an electron beam spot. For the best results, a size of the electron beam spot may preferably be at most 150 μm in diameter, preferably at most 75 μm in diameter, and more preferably at most 50 μm in diameter. The method may enable high productivity of microstructures 10 with high density septa walls 20 for selective transmission of X-ray or gamma-ray radiation. The structures may be built without support structures and have little or no micro-cracking and excellent surface properties compared to previously known manufacturing technologies. Thanks to the very high material density (high-Z material), in combination with the small electron beam spot size, the method enables high resolution manufacturing of the microstructures. Thin layers of the powder, e.g. in combination with small particle size, may also enable high resolution manufacturing of high density septa walls 20.

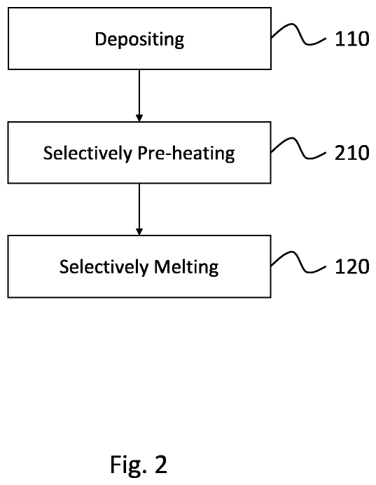

FIG. 2 illustrates a method similar to the method in FIG. 1, with an additional step 210 of selectively pre-heating spots of the powder before the powder is selectively melted and solidified 120 in a spot sequence. In this example, spots 40 of the powder are selectively pre-heated 210 by scanning the electron beam in a spot pre-sintering sequence on the layer of powder. The spot pre-sintering sequence is thus carried out before the spot sequence. By selectively and accurately pre-heating or pre-sintering spots 40 of powder with the electron beam, subsequent melting of the powder with the spot-sequence can be improved. The scanning direction and sequence of spots of the pre-sintering sequence may be the same as a scanning direction and sequence of spots of the spot sequence for all spots 40 or for a plurality of spots 40.

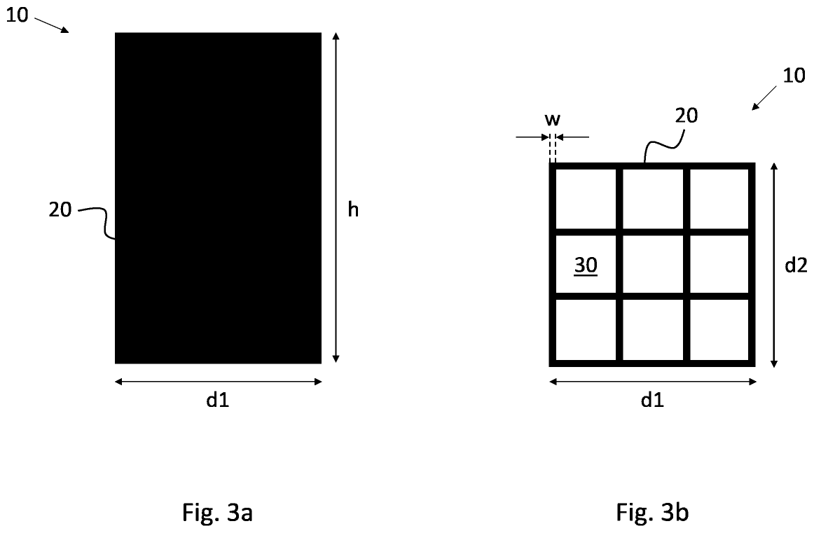

FIG. 3a and FIG. 3b schematically show a microstructure 10 manufactured with the method according to embodiments of the present invention. The microstructure 10 enables selective transmission of X-ray or gamma-ray radiation and may therefore advantageously be used in an imaging component, such as an X-ray or gamma-ray filter, an X-ray or gamma-ray collimator, an X-ray or gamma-ray anti-scatter device, and/or an X-ray or gamma-ray grating. The microstructure 10 may comprise septa wall structures 20 in one or in multiple directions. In the example in FIG. 3a and FIG. 3b, the microstructure 10 is a grid-like structure with septa walls 20 in a first direction d1 and a second direction d2. Such a structure may e.g. be used for two-dimensional anti-scatter grids. In the example in FIG. 3a and FIG. 3b, the angle between septa walls 20 in the first direction d1 and second direction d2 is about 90 degrees. However, other structures with different angles between the walls, such as but not limited to honeycomb like structures, are also conceivable. FIG. 3a shows a side view of the example microstructure 10. In this case the septa wall 20 seen from the side-view of FIG. 3a has a length in first direction d1 and a height h. FIG. 3b illustrates the same structure 10 from above. As seen from the figure, the microstructure 10 has septa walls 20 in the direction of d1 and, in this case orthogonally, in the direction of d2.

The walls have a thickness w. In FIG. 3 b, all septa walls have the same thickness w, but it is also possible that different walls have different thickness. The septa walls 20 are dense structures, that may be able to efficiently absorb or reflect radiation. The space between the walls forms transmissive sections 30, which lets radiation pass. With the manufacturing method according to embodiments of the present invention, it is possible to manufacture dense and thin septa walls 20. The septa walls may have a wall thickness w of at most 200 μm, preferably at most 100 μm, or more preferably at most 70 μm. Each septa wall 20 has a height h and a thickness w. The septa walls 20 may have a height h to thickness w aspect ratio of at least 100, preferably at least 200, and more preferably at least 400. For example, a septa wall 20 for an anti-scatter grid may advantageously have a thickness w of 70-100 μm and a height h of 40-50 mm. Such microstructures 10 with a large height h to thickness w ratio may enable precise control of transmission of X-ray or gamma-ray radiation through the transmissive sections 30 while having low losses in radiation intensity due to thickness w of the walls 20. For production of imaging components with such microstructures 10, multiple microstructures 10 may be stacked on top of each other and/or tiled together in order to achieve components with desired specification of total height, area etc.

FIG. 4a and FIG. 4b schematically illustrate spot sequences for additive manufacturing of a microstructure for selective transmission of X-ray or gamma-ray radiation, according to an embodiment of the invention. In each spot sequence a plurality of spots 40 on the layer of high-Z material powder are sequentially exposed to an electron beam spot to melt and solidify the powder during the process to form septa wall structures 20. FIG. 4a illustrates spot sequences in the direction d1 and FIG. 4b illustrates spot sequences in the direction d2. The example microstructure 10 in FIG. 4a and FIG. 4b has three septa walls 20 parallel to the direction d1 and three septa walls parallel to the direction d2. In this particular example, dl is orthogonal to d2 but angles other than 90 degrees between the directions are also possible, depending on the specifications of the microstructure 10 at hand. A total spot sequence to melt (fully or partially) and solidify the powder to build a layer of each of the three-by-three septa walls 20 in FIG. 4a and FIG. 4b may comprise three sequences parallel to the d1 direction and three sequences parallel to the d2 direction. For example, the sequence may begin with three paths along the three septa walls 20 in the d1 direction, such as in FIG. 4a A to B, C to D and E to F, followed by three sequential paths parallel to the direction d2, such as in FIG. 4b G to H, I to J and K to L. For each path, a plurality of spots 40 are sequentially exposed to the electron beam. The electron beam current may be e.g. 10-100 mA and the scanning speed e.g. up to or even larger than 2 m/s. The spot sequence strategy may enable to achieve a desirable grain structure (equiaxed/columnar) of the manufactured septa walls 20. In addition, the amount of sintered powder within a transmissive section (pixel) 30 of the microstructure 10 may be reduced.

Different options of the spot sequence may be considered, such as in FIG. 4a the individual paths may be either from right to left or from left to right figure. Similarly in FIG. 4b the individual paths may be either top to bottom or bottom to top in the figure. For illustration, non-limiting examples of sequences in the d1 direction may be, A to B, D to C and E to F, or F to E, C to D, B to A, or E to F, C to D and A to B, etc. Similarly, different variations top to bottom or bottom to top of the paths in FIG. 4b may be considered. Such dedicated spot melting strategies may reduce the amount of sintered powder within a pixel of a grid to enable high resolution. Furthermore, the strategy may be used to achieve a desirable grain structure in each of the septa walls 20.

In another example, the sequences in FIG. 4a and FIG. 4b may be carried out in an alternating fashion with one sequence in the d1 direction followed by a sequence in the d2 direction. Such as e.g., but not limited to, A to B followed by G to H, C to D followed by I to J, and E to F followed by K to L. Also in this case, many variations of the direction and/or order of sequences are conceivable.

It is noted that the sequences in FIG. 4a and FIG. 4b are only drawn schematically. Any sequence in the d1 or d2 direction may be shorter or longer than what is illustrated in the figures. As a non-limiting example, a sequence in the d1 or d2 direction may be shortened and/or skip a spot 40 such that it does not repeat melting in e.g. spots 40 that are melted in a respective d2 or d1 sequence.

In an example of a method including an additional step 210 of selectively pre-heating spots 40 of the powder before the powder is selectively melted and solidified 120 in a spot sequence, it may be advantageous that a scanning direction and sequence of spots of the pre-sintering sequence is the same or similar as a scanning direction and sequence of spots of the spot sequence. That is, with reference to the example in FIG. 4a and FIG. 4b, to follow the same scanning sequences described above also for selectively pre-heating spots 40 of the powder. This may be the case for all spots 40, or that a scanning direction and sequence of spots of the pre-sintering sequence is the same or similar as a scanning direction and sequence of spots of the spot sequence for a plurality of spots 40. Pre-heating and melting the powder according to the same or partly the same pattern, may be advantageous for the formation of septa walls 20 with high density. Manufacturing in this way to achieve septa walls 20 with a high density may be particularly advantageous for manufacturing of a microstructure 10 for selective transmission of X-ray or gamma-ray radiation, such as structures included in e.g. 2D anti scatter grids.

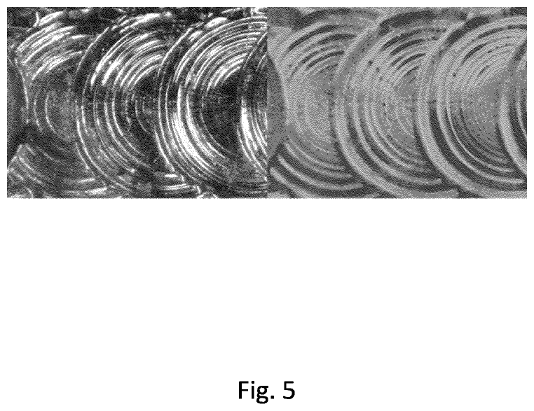

FIG. 5 shows optical surface images of a septa wall produced by a method of additive manufacturing according to an embodiment of the invention. The left and right side of the figure show the same part of the surface under different lighting conditions of an optical microscope. The images clearly illustrate the pattern of individually exposed spots 40 as part of a spot melting strategy.

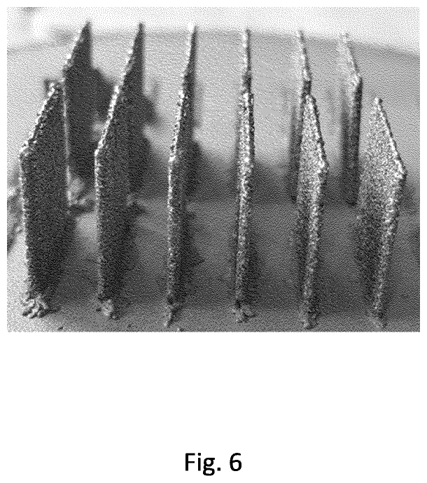

FIG. 6 shows an image of individual septa walls produced by a method of additive manufacturing according to an embodiment of the invention. In this case, multiple walls in a first direction, such as in d1, have been manufactured.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The notion “at most” means “equal to or less than”, and the notion “at least” means “equal to or more than”. The invention may be implemented by means of hardware comprising several distinct elements, and/or by means of a suitably programmed processor. In the device claim enumerating several means, several of these means may be embodied by one and the same item of hardware. Measures recited in mutually different dependent claims may advantageously be used in combination.

1. A method for additive manufacturing of a microstructure, the method comprising:
depositing a layer of powder on a build plate or on a layer from at least one previous manufacturing step on the build plate, wherein the layer of powder comprises a high-Z material powder, wherein the high-Z material powder comprises a high-Z material; and
selectively melting and solidifying the powder to form septa wall structures by scanning an electron beam in a spot sequence on the layer of powder to sequentially expose the layer of powder to a plurality of electron beam spots, wherein a size of the electron beam spots is at most 75 μm in diameter, in diameter,
wherein the septa wall structures have a wall thickness of at most 100 μm, and
wherein the septa wall structures have a height to thickness aspect ratio of at least 100.

2. The method according to claim 1, wherein the high-Z material powder comprises at least one of Tungsten, Molybdenum, Tantalum, Niobium, Lead, Bismuth, Rhenium, Silver, and Gold.

3. The method according to claim 1, wherein the high-Z material powder has a particle size distribution with a median diameter of at most 50 μm.

4. The method according to claim 1, wherein the deposited layer of powder has a layer thickness between 10 μm and 50 μm.

5. The method according to claim 1, further comprising selectively pre-heating spots of the powder by scanning the electron beam in a spot pre-sintering sequence on the layer of powder, and wherein the spot pre-sintering sequence is carried out before the spot sequence.

6. The method according to claim 5, wherein a scanning direction and sequence of spots of the pre-sintering sequence is the same as a scanning direction and sequence of spots of the spot sequence for all spots, or wherein a scanning direction and sequence of spots of the pre-sintering sequence is the same as a scanning direction and sequence of spots of the spot sequence for a plurality of spots.

7. The method according to claim 1, wherein the septa wall structures of the microstructure comprise first wall structures formed with each respective length axis parallel to a first direction and second wall structures formed with each respective length axis parallel to a second direction, and wherein the second direction is at an angle to the first direction.

8. The method according to claim 7, wherein the spot sequence to sequentially expose a plurality of spots on the layer of powder to the electron beam spot comprises a first sequence followed by a second sequence, wherein the first sequence comprises scanning the electron beam spot to sequentially expose a plurality of spots on the length axes parallel to the first direction, and wherein the second sequence comprises scanning the electron beam spot to sequentially expose a plurality of spots on the length axes parallel to the second direction.

9. The method according to claim 8, wherein the first sequence comprises scanning the electron beam along a plurality of first septa wall paths on the respective length axes parallel to the first direction, and wherein the second sequence comprises scanning the electron beam along a plurality of second septa wall paths on the respective length axes parallel to the second direction.

10. The method according to claim 1, wherein the septa wall structures have a wall thickness of at most 200 μm.

11. (canceled)

12. (canceled)

13. An imaging component comprising a microstructure for selective transmission of X-ray or gamma-ray radiation, wherein the microstructure is obtained by the method according to claim 1.

14. The imaging component according to claim 13, wherein the imaging component comprises a plurality of stacked microstructures.

15. The imaging component according to claim 13, wherein the imaging component comprises one or more of:
an X-ray or gamma-ray filter;
an X-ray or gamma-ray collimator;
an X-ray or gamma-ray anti-scatter device; and
an X-ray or gamma-ray grating.

16. A method for manufacturing an imaging component, comprising:
manufacturing at least one microstructure comprising:
depositing a layer of powder on a build plate or on a layer from at least one previous manufacturing step on the build plate, wherein the layer of powder comprises a high-Z material powder, wherein the high-Z material powder comprises a high-Z material; and
selectively melting and solidifying the powder to form septa wall structures by scanning an electron beam in a spot sequence on the layer of powder to sequentially expose the layer of powder to a plurality of electron beam spots, wherein a size of the electron beam spots is at most 75 μm in diameter in diameter, wherein the septa wall structures have a wall thickness of at most 100 μm, and wherein the septa wall structures have a height to thickness aspect ratio of at least 100; and
forming the imaging component from the at least one microstructure

17. The method according to claim 16, wherein forming the imaging component comprises stacking a plurality of the microstructures.

18. The method according to claim 16, wherein the imaging component comprises one or more of:
an X-ray or gamma-ray filter;
an X-ray or gamma-ray collimator;
an X-ray or gamma-ray anti-scatter device; and
an X-ray or gamma-ray grating.
